(19) United States
(12) Patent Application Publication
(10) Pub. No.: US 2026/0306860 A1
(43) Pub. Date: Oct. 8, 2026

(54) EXTENDING SERVICE TIME OF IMPLANTABLE CARDIOVERTER DEFIBRILLATORS

(71) Applicant: Pacesetter, Inc., Sylmar, CA (US)

(72) Inventors: David Bowen, Taylors, SC (US); Ralph Jason Hemphill, Sunset, SC (US); Abdul Nasir Arghandehwal, Simi Valley, CA (US); Sriveni Muddani, Pacoima, CA (US); Brian Greig, Phoenix, AZ (US); Nicole Cooper, Burbank, CA (US); Xiaofei Jiang, Clemson, SC (US)

(21) Appl. No.: 19/481,202

(22) PCT Filed: Apr. 5, 2024
(86) PCT No.: PCT/US2024/023396

§ 371 Date: Nov. 4, 2025

Related U.S. Application Data
(60) Provisional application No. 63/464,526, filed on May 5, 2023.

(51) Int. Cl. A61N 1/39; A61N 1/378

(52) U.S. Cl. CPC A61N 1/3956; A61N 1/3787; A61N 1/3975

(57) ABSTRACT

An implantable cardioverter-defibrillator (ICD) includes one or more capacitors, a secondary battery and a primary battery. The implantable cardioverter-defibrillator also includes a capacitor charging circuit. A controller operates the capacitor charging circuit such that the one or more capacitors are charged with electrical energy from the secondary battery or the primary battery. In some instances, the controller selects whether the capacitors are charged with electrical energy from the secondary battery or the primary battery. In some instances, the one or more capacitors are charged with electrical energy from the secondary battery and the ICD includes a charging circuit that the controller operates such that electrical energy from the primary battery recharges the secondary battery.

### RELATED APPLICATIONS

This application is a continuation of U.S. patent application Ser. No. 63/464,526, filed May 5, 2023, entitled “Extending Service Time of Implantable Cardioverter Defibrillators”, and incorporated herein in its entirety.

### FIELD

The invention relates to medical devices. In particular, the invention relates to implantable cardioverter-defibrillators.

### BACKGROUND

Traditional implantable cardioverter-defibrillators (ICD) are powered by primary batteries. As a result, ICDs have a finite amount of battery capacity and accordingly a finite service time before surgical intervention is needed to replace the batteries or the ICD. Currently, ICDs have a service time of around 7-12 years. An ICD that provides an extended service time would be valuable to clinicians/patients. As a result, there is a need for an improved implantable cardioverter-defibrillator (ICD).

### SUMMARY

An implantable cardioverter-defibrillator (ICD) includes one or more capacitors, a secondary battery, and a primary battery. The implantable cardioverter-defibrillator also includes a charging circuit and a capacitor charging circuit. A controller operates the capacitor charging circuit such that the one or more capacitors are charged with electrical energy from the secondary battery. Additionally, the controller operates the charging circuit such that electrical energy from the primary battery recharges the secondary battery.

Another embodiment of an implantable cardioverter-defibrillator (ICD) includes one or more capacitors, a capacitor charging circuit, and multiple batteries. A controller operates a switch circuit so as to select a portion of the batteries to act as a source for electrical energy that charges the one or more capacitors. The controller performs capacitor charges. During each of the capacitor charges, the controller operates the capacitor charging circuit such that the one or more capacitors are charged with electrical energy from the selected portion of the batteries.

Another embodiment of an implantable cardioverter-defibrillator (ICD) includes one or more capacitors. The ICD also includes telemetry receiver configured to be in wireless communication with a telemetry transmitter located outside of a patient while the ICD is located inside of the patient. The ICD has a controller configured to perform a maintenance charge of the one or more capacitors in response to the telemetry receiver receiving from the telemetry transmitter a request that the controller perform the maintenance charge. Electrical energy charges the one or more capacitors during the maintenance charge. In some instances, a telemetry transceiver serves as the telemetry receiver.

A method of operating an implantable cardioverter-defibrillator (ICD) includes transmitting a signal from a telemetry transmitter located outside of a patient to the ICD while the ICD is located inside of the patient. The signal causes the ICD to perform a maintenance charge of one or more capacitors included in the ICD. The method also includes transmitting electrical energy from a wireless power transfer transmitter to a wireless power transfer receiver included in the ICD while the ICD is located inside of the patient. The ICD employs the electrical energy received from the wireless power transfer transmitter to recharge a secondary battery included in the ICD. The signal and the electrical energy are transmitted during the same visit of the patient to a treatment clinic.

### BRIEF DESCRIPTION OF THE FIGURES

FIG. 1 is a block diagram of an example of an implantable cardioverter-defibrillator (ICD).

FIG. 2 is a block diagram of another example of an Implantable Cardioverter-Defibrillator (ICD).

FIG. 3 is a schematic of an example of a capacitor charging circuit that is suitable for use in the Implantable Cardioverter-Defibrillator (ICD).

FIG. 4A is a schematic of an example of a capacitive charge pump suitable for use as the primary battery charging circuit.

FIG. 4B is a schematic of an example of a capacitive charge pump suitable for use as the primary battery charging circuit.

FIG. 5 is a schematic of an example of a wireless power transfer system that includes an example of a suitable wireless power transfer receiver and an example of a suitable wireless power transfer transmitter

FIG. 6 is a schematic that includes a schematic of an example of a sensing circuit.

FIG. 7 is a diagram of an example of a pulse generator for use in an ICD.

### DESCRIPTION

An ICD includes a primary battery and a secondary battery. While implanted, the secondary battery can be recharged by a wireless power transfer transmitter. As a result. a portion of the electrical energy expended during operation of an implanted ICD can be replaced. The ability to replace a portion of the energy expended by an implanted ICD extends the time that the ICD provides service before battery replacement is needed.

The ICD is configured to perform capacitor charges where one or more capacitors are charged by the ICD. In some instances, the ICD discharges the one or more capacitors so the electrical energy stored in the one or more capacitors is delivered to the patient as a therapeutic shock. The ICD can use electrical energy stored in the secondary battery to charge the one or more capacitors for at least a portion of the capacitor charges. Secondary batteries can generally be discharged at higher rates than primary batteries. As a result, charging the one or more capacitors with a secondary battery can increase the efficiency of charging the one or more capacitors.

In one embodiment, the energy stored in the secondary battery is used to charge the one or more capacitors and is also used for the background operations of the ICD. Additionally, the primary battery recharges the secondary battery so as to keep the secondary battery within a characteristic window such as a state of charge window. Further, the ICD can be constructed such that energy stored in the primary battery does not directly charge the one or more capacitors. As a result, the primary battery need not have high-rate capabilities. Since the importance of the high-rate capabilities for the primary battery is reduced, the primary battery can be selected to provide enhanced safety to the patient and/or to have an increased energy density. This increase in energy density can further extend the time that the ICD provides service before battery replacement is needed.

The ICD can charge the one or more capacitors in response to the ICD finding that charge criteria have been satisfied. Additionally, the ICD can refrain from charging the one or more capacitors in response to the ICD finding that charge criteria have not been satisfied. However, different charges of the one or more capacitors can be done in response to satisfaction of different charge criteria such as treatment criteria, backup criteria, and/or maintenance criteria. As an example, the ICD can charge the one or more capacitors in response to the ICD detecting a cardiac arrhythmia suitable for treatment by a therapeutic shock from the ICD (satisfaction of one or more treatment criteria); in response to the ICD detecting that a prior therapeutic shock has not restored normal rhythm to the heart (satisfaction of one or more backup criteria); or in response to the ICD determining that maintenance of the one or more capacitors should be performed (satisfaction of one or more maintenance criteria).

The ICD can select which battery charges the one or more capacitors in response to the charge criteria that the ICD finds to be satisfied. In one embodiment, the ICD uses the primary battery to charge the one or more capacitors when the ICD detects the presence of a cardiac arrhythmia suitable for treatment by a therapeutic shock from the ICD. Additionally, the ICD uses the secondary battery to charge the one or more capacitors in response to the ICD determining that a prior therapeutic shock has not restored normal rhythm to the heart and/or in response to the ICD determining that that maintenance of the one or more capacitors should be performed. Additionally, the secondary battery can also provide the energy for background functions such as detection of cardiac arrhythmias. Since the energy expended from the secondary battery can be replaced by recharging the secondary battery the capacity of the primary battery can determine the time the ICD is in service before surgical intervention is needed to replace or maintenance the ICD. Since the energy from the primary battery is primarily used to provide primary therapeutic shocks that respond to the detected cardiac arrhythmia the frequency of primary therapeutic shocks can determine the service time of an implanted ICD. As a result the primary battery can be selected to provide at least the number of primary therapeutic shocks that are anticipated before surgery for maintenance of the ICD is required.

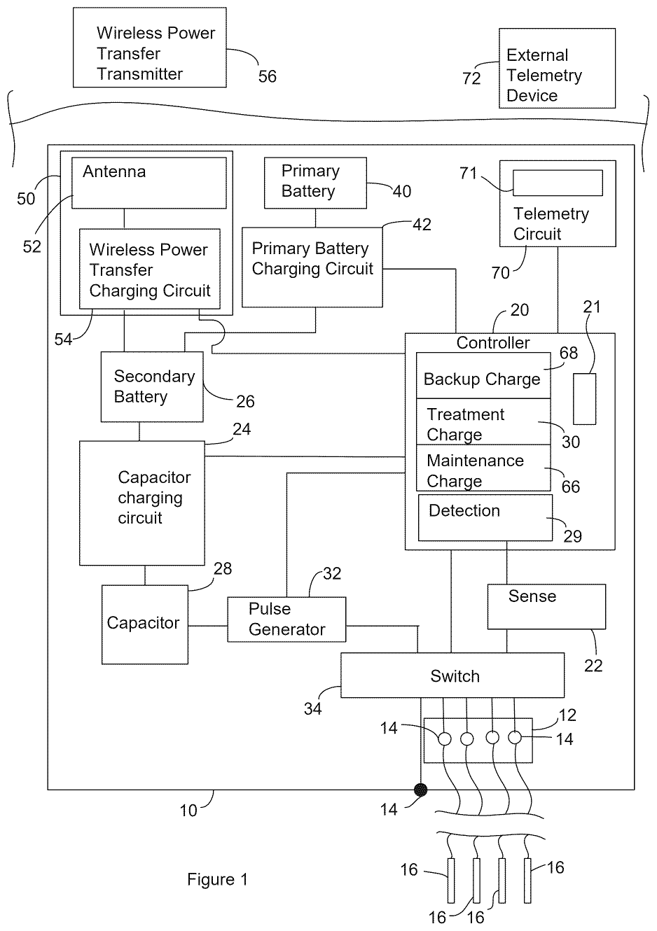

FIG. 1 is a block diagram of an implantable cardioverter-defibrillator (ICD) that includes multiple batteries that can consist of or include a primary battery and a secondary battery. The ICD can be a transvenous ICD or a subcutaneous ICD. The implantable cardioverter-defibrillator (ICD) can be configured to detect the occurrence of cardiac arrhythmias such as ventricular tachycardia and ventricular fibrillation. The ICD can also be configured to provide therapeutic shocks in response to the detected arrhythmia. The timing and waveform of the therapeutic shocks can be selected to restore the normal rhythm to the heart.

The ICD includes a pulse generator 8 that can be implanted in a patient. The pulse generator 8 includes a connector block 12 having one or more terminals 14. Each of the terminals 14 can be electrically coupled with one or more electrodes 16. Each of the electrodes 16 can be used for applying a therapeutic shock to a patient and/or for sensing electrical properties that indicate the presence of a cardiac arrhythmia. A lead 18 can provide an electrical coupling between each of the terminals 14 and one or more of the electrodes 16. In some instances, the housing 10 serves as a terminal 14 and an electrode 16. For instance, the housing 10 can serve as a return electrode for all or a portion of the other electrodes 16. As is known in the ICD arts, the electrodes 16 can be placed in and/or near the patient's heart as needed to detect the presence of a cardiac arrhythmia and/or deliver therapeutic shocks that treat the detected arrhythmia.

The ICD includes a controller 20 that can optionally be programmable. Suitable controllers 20 can include components such as analog electrical circuits, digital electrical circuits, Application Specific Integrated Circuits (ASICs), processors, microprocessors, digital signal processors (DSPs), Field Programmable Gate Arrays (FPGAs), computers, microcomputers, or combinations suitable for sensing the presence of an arrhythmia and delivery of the therapy in response to a sensed arrhythmia. In some instances, the controller can include and/or access a storage device 21 such as a RAM or ROM memory.

The ICD includes one or more sensing circuits 22 that are each electrically coupled with the controller 20. Each sensing circuit is configured to receive electrical signals from one or more of the electrodes 16. The electrical signals can be representative of electrical behavior of the heart. Each sensing circuit outputs data signals that are received by the controller. Each sensing circuit can process the electrical signals such that the data signals are in a form that can be processed by the controller.

The controller 20 includes a detection module 29 that receives the data signals from the one or more sensing circuits 22. The detection module 29 can be configured to monitor the electrical properties of the heart. The detection module 29 can also be configured to and to detect occurrence of an arrhythmia that can be treated by a therapeutic shock from the ICD. For instance, the detection module 29 can perform one or more sensing algorithms and/or one or more detection algorithms on the data signals. Sensing algorithms can be configured to detect the occurrence of an arrhythmia that can be treated by the ICD and detection algorithms can discriminate between the different classes of arrhythmia. In some instances, detection algorithms can discriminate between the different classes of tachycardia such as supraventricular tachycardia (SVTs) and ventricular tachycardia (VT). Although the one or more sensing circuits 22 are disclosed as being a separate component from the controller, all or a portion of the one or more sensing circuits 22 can be included in the controller 20.

The ICD includes a capacitor charging circuit 24, a secondary battery 26 and one or more capacitors 28. The capacitor charging circuit 24 provides an interface between a secondary battery 26 and the one or more capacitors 28. Suitable capacitors include, but are not limited to, electrolytic capacitors. In some instances where the ICD includes multiple capacitors, the capacitors are connected in parallel.

The controller 20 can include a treatment module 30 that performs a treatment charge of the one or more capacitors in response to the detection module 29 detecting a cardiac arrhythmia suitable for treatment by a therapeutic shock from the ICD. For instance, the controller 20 can operate the capacitor charging circuit 24 such that electrical energy from the secondary battery 24 charges the one or more capacitors 26 in response to the detection module 29 detecting a cardiac arrhythmia suitable for treatment by a therapeutic shock from the ICD. The treatment module 30 can also operate a switch 34 such that at least a portion of the electrical energy stored in the one or more capacitors is received at one or more of the electrodes in the form of a primary therapeutic shock that is delivered to the patient's body The controller 20 operates the switch 34 such that the primary therapeutic shock is received at the electrodes with the desired timing, electrical characteristics, and waveform for treatment of the arrhythmia.

The ICD optionally operates the switch 34 so as to control which terminals are electrically coupled with the one or more capacitors 28 and which terminals are electrically coupled with the one or more sensing circuits 22. As a result. the controller 20 can select which electrodes 16 are connected to the one or more capacitors 28 and which electrodes are connected to the one or more sensing circuits 22. Accordingly a controller can select which of the electrodes deliver a therapeutic shock. Additionally, a terminal 14 and one or more of the electrodes 16 can be connected to the one or more capacitors 28 and all or a portion of the one or more sensing circuits 22. Accordingly, an electrode 16 can be used for sensing and detection applications and/or delivery of therapeutic shocks. A suitable switch 34 includes, but is not limited to, a bridge such as an H-bridge or a push-pull bridge.

The ICD includes a primary battery 40 and a primary battery charging circuit 42. The primary battery charging circuit 42 provides an interface between the primary battery 40 and the secondary battery 26. The controller 20 can operate the primary battery charging circuit 42 such that electrical energy from the primary battery 40 charges the secondary battery 26. In some instances, the beginning of service voltage of the primary battery 40 is below the voltage of the secondary battery 26 when at a 100% state of charge. In the ICD embodiment of FIG. 1, a suitable voltage for the primary battery 40 at beginning of service includes, but is not limited to, a voltage greater than 1.5V, 2.0V, or 2.2V and less than 4.35V, 4.25V, or 3.25V and a suitable fully charged voltage for the secondary battery at beginning of service includes, but is not limited to, a voltage greater than 3.2V, 3.5V or 3.7V and less than 4.35V, 4.25V or 4.0V. Suitable primary battery charging circuits 42 include. but are not limited to, boost converters including synchronous boost converters and capacitive charge pumps.

The operation of the primary battery charging circuit 42 so as charge the secondary battery 26 with energy from the primary battery 40 can be done so as to maintain the secondary battery 26 within a characteristic window. For instance, the controller 20 can operate the primary battery charging circuit 42 so the secondary battery 26 maintains a state of charge within a state of charge window that extends from a lower threshold to an upper threshold. As an example, the controller 20 can operate the primary battery charging circuit 42 such that electrical energy from the primary battery 40 charges the secondary battery 26 in response to the state of charge of the battery falling below the lower threshold. Additionally or alternately, the controller 20 can operate the primary battery charging circuit 42 such that electrical energy from the primary battery 40 stops charging the secondary battery 26 in response to the state of charge increasing above the upper threshold. In some instances, the lower threshold is a state of charge greater than 20%, 30%, or 35% and less than 50%, or 60% and the upper threshold is a state of charge greater than 50%, and less than or equal to 90%, or 100%.

The ICD includes a wireless power transfer receiver 50. The wireless power transfer receiver 50 includes an antenna 52 and a wireless power transfer charging circuit 54. Suitable antenna 52, include, but are not limited to, coils, inductive antennas, radiofrequency (RF) antennas, and blue tooth antennas.

The wireless power transfer receiver 50 can receive power from a wireless power transfer transmitter 56 located outside of the patient's body 58. The wireless power transfer transmitter 56 can have its own power source such as a battery or can receive power from an external source such as a wall socket The wireless power transfer transmitter 56 can be a stand-alone component or can be included in or on an article of clothing or equipment worn by the patient. For instance, the wireless power transfer transmitter 56 can be included in or on a vest worn by the patient. When the wireless power transfer transmitter 56 is included in or on an article of clothing or equipment worn by the patient, the article of clothing or equipment can hold the wireless power transfer transmitter 56 in an appropriate location relative to the wireless power transfer receiver 50 to establish a wireless power transfer link by which wireless power transfer can occur between the wireless power transfer receiver 50 and the wireless power transfer transmitter 56.

The wireless power transfer receiver 50 provides an interface between the wireless power transfer receiver 50 and the secondary battery 26. The controller 20 can operate the wireless power transfer charging circuit 54 such that the electrical energy that the wireless power transfer receiver 50 receives from the wireless power transfer transmitter 56 charges the secondary battery 26. For instance, when there is a wireless power transfer link by which wireless power transfer can occur between the wireless power transfer receiver 50 and the wireless power transfer transmitter 36, the controller 20 can operate the wireless power transfer charging circuit 54 so as to charge the secondary battery 26 with energy from the wireless power transfer transmitter 56. In some instances, the controller 20 can operate the wireless power transfer charging circuit 34 so as to charge the secondary battery 26 with energy from the wireless power transfer transmitter 56 in response to the wireless power transfer receiver 50 being positioned sufficiently close to the wireless power transfer transmitter 56 to establish the wireless power transfer link. Accordingly, the wireless power transfer receiver 50, wireless power transfer transmitter 56 and secondary battery 26 can operate as a wireless power transmission system where the wireless power transfer transmitter 56 recharges the secondary battery 26 through the use of protocols such as Wireless power transfer (WPT), wireless power transmission, wireless energy transmission (WET), or electromagnetic power transfer. In one example, the wireless power transmission system transfer is configured such that the wireless power transfer transmitter 56 generates a time-varying electromagnetic field, which transmits power through the body of the patient to the wireless power transfer receiver 50 in the implanted ICD, which extracts power from the field and supplies it to the secondary battery 26. In some instances, the power is transmitted through the body of the patient to the wireless power transfer receiver 50 by inductive coupling including resonant inductive coupling.

When the controller 20 operates the wireless power transfer charging circuit 54 so as to charge the secondary battery 26 with energy from the wireless power transfer transmitter 56, the controller 20 can operate the wireless power transfer charging circuit 54 so as to charge the secondary battery 26 to a state of charge within the characteristic window such as the state-of-charge window. For instance, the controller 20 can operate the wireless power transfer charging circuit 54 so as to charge the secondary battery 26 to a state of charge greater than 50%, and less than or equal to 100%. If a user frequently charges the secondary battery 26 with energy from the wireless power transfer transmitter 56, charging the secondary battery 26 with energy from the primary battery 40 may not occur and/or be needed. In some instances, the secondary battery 26 can be concurrently charged with electrical energy from the primary battery 40 and with electrical energy from the wireless power transfer transmitter 56 through concurrent operation of the wireless power transfer charging circuit 54 and the primary battery charging circuit 42.

The ICD includes a telemetry circuit 70 that can operate as one or more of a telemetry receiver, telemetry transceiver, or telemetry transmitter The telemetry circuit 70 can include an antenna 71 that can be in telemetric communication with an external telemetry device 72 located outside of the patient's body. The controller 20 can operate the telemetry circuit 70 so as to transmit and/or receive data, programming instructions, and/or status information relating to the operation of the device with the external telemetry device 72.

Additionally or alternately, the controller 20 can receive instructions from the external telemetry device. The controller 20 can execute the instructions in response to receipt of the instruction. For instance, the controller can receive an instruction to perform a maintenance charge the external telemetry device. The controller can perform the maintenance charge in response to the received instruction. The controller 20 can store received data, programming instructions, and/or status information in the storage device 21. Additionally or alternately, the controller 20 can transmit data, programming instructions, and/or status information stored in the storage device 21. As a result, the telemetry circuit 70 can be used to program the ICD or change operating parameters of the ICD. Suitable antenna 71 include, but are not limited to, coils, inductive antennas, radiofrequency (RF) antennas, and blue tooth antennas.

The controller 20 can include multiple different charging modules that each performs a different type of capacitor charge. Examples of different types of capacitor charges include treatment charges, maintenance charges, and backup charges. Each type of capacitor charge being associated with one or more charge criteria. The one or more charge criteria associated with the capacitor charges are different for different types of capacitor charges. The controller can perform each type of capacitor charge in response to satisfaction of the one or more criteria associated with the type of capacitor charge. As a result, different capacitor charges can be done in response to satisfaction of different charging criteria.

The treatment module 30 disclosed above is an example charging module. The treatment module 30 performs a treatment charge where the treatment module charges the one or more capacitors 26 in response to the detection module 29 detecting a cardiac arrhythmia suitable for treatment by a therapeutic shock from the ICD. The detection module 29 applies one or more sensing algorithms and/or one or more detection algorithms to data signals in order to identify the occurrence of the cardiac arrhythmia. The one or more sensing algorithms and/or one or more detection algorithms include one or more charging criteria that are satisfied in order for the detection module 29 to identify that a cardiac arrhythmia has occurred or is occurring. The actual charging criteria that are satisfied in order for the detection module 29 to identify occurrence of the cardiac arrhythmia is a function of the one or more sensing algorithms and/or one or more detection algorithms that are performed.

The charging modules can also include a maintenance module 66 that performs a maintenance charge in response to satisfaction of one or more maintenance criteria. For instance, the maintenance module 66 can operate the capacitor charging circuit 24 such that electrical energy from the secondary battery 24 charges the one or more capacitors 26 in response to the controller 20 detecting satisfaction of one or more maintenance criteria. Each of the one or more maintenance criteria can serve as a charging criterion. The one or more maintenance criteria can include a time threshold. The time threshold can be a threshold in the time that has passed since the last time the one or more capacitors 26 were charged. Capacitors, such as electrolytic capacitors, can experience deformation when they are not charged over time. The deformation can be a result of the electrolyte within the capacitor chemically attacking an oxide formed on the electrodes of the capacitor. The deformation can be prevented or reduced by charging the capacitor. The charging of the capacitor restores the quality of the oxide on the capacitor electrodes. The maintenance module 66 can include or access a time measurement device such as a clock or other counter to determine the time that has passed since the one or more capacitors 26 were last charged. The maintenance module 66 can determine that the one or more maintenance criteria are satisfied when the time since the one or more capacitors 26 were last charged exceeding the time threshold Accordingly, the maintenance module 66 can operate the capacitor charging circuit 24 such that electrical energy from the secondary battery 24 charges the one or more capacitors 26 in response to the time since the one or more capacitors 26 were last charged exceeding the time threshold.

In one example of a maintenance charge, the one or more capacitors 26 are charged to at least a treatment charge voltage one or more times. The treatment charge voltage is at least equal to the voltage to which the one or more capacitors are charged during a treatment charge. When the maintenance charge includes multiple charges to the treatment charge voltage, the value of the treatment charge voltage for different charges to the treatment charge voltage can be the same or different. Additionally, when the maintenance charge includes multiple charges to the treatment charge voltage, adjacent charges to the treatment charge voltage can be separated and/or followed by a pause time where the voltage of the one or more capacitors 26 is allowed to drop below the treatment charge voltage. When the voltage of the one or more capacitors is allowed to drop below the treatment charge voltage between adjacent charges to the treatment charge voltage, the voltage of the one or more capacitors can be allowed to drop to a voltage that is less than 50% or 90% of the treatment charge voltage. In some instances, the one or more charges to the treatment charge voltage includes holding the one or more capacitors at the treatment charge voltage for a charge hold time.

The maintenance charge can optionally include a nominal charge before the one or more charges of the one or more capacitors to the treatment charge voltage. During the nominal charge, the one or more capacitors 26 are charged to a nominal voltage. The nominal voltage can be less than the treatment charge voltage. In some instances, the nominal voltage is greater than 30%, or 50% of the treatment charge voltage and less than 95% of the treatment charge voltage. The charge to the nominal voltage can be followed by a pause time where the voltage of the one or more capacitors 26 is allowed to drop from the nominal voltage. In some instances, the charge to the nominal voltage includes holding the one or more capacitors at the nominal voltage for a charge hold time.

The presence of the charge hold time(s) and/or the use of multiple charges to the treatment charge voltage repairs the oxide coating on the electrode of capacitors such as electrolytic capacitors and reduces or prevents deformation. In some instances, suitable pause times for charges to a nominal voltage or a treatment charge voltage include, but are not limited to, times greater than or equal to 2 seconds, or 1 minute and less than or equal to 10 minutes. Different pause times during the same maintenance charge can be the same or different. In some instances, suitable charge hold times for charges to a nominal voltage or a treatment charge voltage include, but are not limited to, times greater than or equal to 1 second and less than or equal to 1 minute or 10 minutes. Different charge hold times during the same maintenance charge can be the same or different.

Rather than operating the switch 34 so as to deliver the electrical energy stored in the one or more capacitors during a maintenance charge to the electrodes 16, the maintenance module 66 can allow the electrical energy stored in the one or more capacitors to dissipate without being delivered to the electrodes 16 and/or without being delivered to the electrodes 16 as part of therapy. For instance, the electrical energy stored in the one or more capacitors during a maintenance charge can be allowed to dissipate into the circuitry of the controller 20, the wireless power transfer receiver 50, and/or the telemetry circuit 70 As an example, the instance, the electrical energy stored in the one or more capacitors during a charge to a nominal voltage and/or one or more charges to the treatment charge voltage can be allowed to dissipate into the circuitry of the controller 20, the wireless power transfer receiver 50, and/or the telemetry circuit 70. A portion of the electrical energy stored in the one or more capacitors during a maintenance charge can also be consumed in the repair of oxide on the electrodes of capacitors such as electrolytic capacitors. Additionally or alternately, the controller can use the electrical energy stored in the one or more capacitors during a maintenance charge to recharge the secondary battery 26. For instance, the controller 20 can include a charging circuit (not shown) and the controller can operate the charging circuit such that the electrical energy stored in the one or more capacitors recharges the secondary battery 26. As a result, during a maintenance charge, the electrical energy stored in the one or more capacitors is not applied to the patient through the one or more electrodes 16 or is not substantially applied to the patient through the one or more electrodes 16. For instance, the electrical energy stored in the one or more capacitors can be allowed to dissipate without being substantially delivered to the electrodes 16 and/or without being delivered to the electrodes 16 as part of therapy. There may be some off-state current leakage from the switch 34, however, the off-state current leakage is generally less than 1 microampere.

The charging modules can also include a backup module 68 that performs a backup charge in response to in response to satisfaction of one or more backup criteria For instance, the backup module 68 can operate the capacitor charging circuit 24 such that electrical energy from the secondary battery 24 charges the one or more capacitors 26 in response to satisfaction of one or more backup criteria. Each of the one or more backup criteria can serve as a charging criterion. Satisfying the one or more backup criteria can indicate whether a prior therapeutic shock that was delivered to the patient has addressed the detected cardiac arrhythmia. The backup module 68 can charge the one or more capacitors 26 in response to the controller 20 detecting that the prior therapeutic shock did not address the detected cardiac arrhythmia. The backup module 68 can also operate the switch 34 such that at least a portion of the electrical energy stored in the capacitor is received at one or more of the electrodes in the form of a backup therapeutic shock that is delivered to the patient's body through one or more of the electrodes 16. The backup module 68 operates the switch 34 such that the backup therapeutic shock is received at the electrodes with the desired timing, electrical characteristics, and waveform for treatment of the arrhythmia. One or more features selected from the group consisting of the timing, waveform, and electrical characteristics of the backup therapeutic shocks can be the same or different from the primary therapeutic shocks. The prior therapeutic shock can be a prior primary therapeutic shock or a prior backup therapeutic shock. In some instances, the backup module 68 responds to detecting that the prior therapeutic shock did not address the detected cardiac arrhythmia by charging the one or more capacitors 26 to the treatment charge voltage, or substantially to the treatment charge voltage, and operating the switch 34 such that the that the backup therapeutic shock is received at the electrodes with the same, or substantially the same, energy as the prior therapeutic shock or the primary therapeutic shock.

As an example of the backup module 68 applying the one or more backup criteria, the backup module 68 can determine that the one or more backup criteria are satisfied when the backup module 68 detects the occurrence of a cardiac arrhythmia that can be treated by a therapeutic shock from the ICD within a backup time from the previously delivered therapeutic shock. In order to detect the occurrence of a cardiac arrhythmia, the backup module 68 can apply one or more sensing algorithms and/or one or more detection algorithms on the data signals. The one or more sensing algorithms and/or one or more detection algorithms can be the same or different from the one or more sensing algorithms and/or one or more detection algorithms applied to the data signals by the detection module 29. When the one or more sensing algorithms and/or one or more detection algorithms are the same as the one or more sensing algorithms and/or one or more detection algorithms applied by the detection module 29, the backup module 68 can use the arrhythmia detection results provided by the detection module 29 rather than performing a separate application of the sensing algorithms and/or detection algorithms. Suitable backup times include, but are not limited to, times greater than or equal 1 second, 2.5 seconds, and less than or equal to 5 minutes.

As an example of the backup module 68 applying the one or more backup criteria, the backup module 68 can determine that the one or more backup criteria are satisfied when the backup module 68 detects that a normal rhythm has not been restored to the heart within a second backup time from the previously delivered therapeutic shock. In order to detect the normal rhythm, the backup module 68 can apply one or more normal rhythm detection algorithms to the data signals.

The one or more capacitors 28 are charged with energy from the secondary battery 26. Prior ICDs charged the one or more capacitors 28 with energy from a primary battery. Secondary batteries can generally be discharged at higher rates than primary batteries. Increasing the rate that the one or more capacitors 28 are charged reduces the increases in Equivalent Series Resistance (ESR) that the one or more capacitors 28 experiences with time. As a result, charging the one or more capacitors 28 with a secondary battery can increase the efficiency of charging the one or more capacitors 28 and can increase the life of the one or more capacitors 28.

Additionally, primary batteries can have a higher energy density than secondary batteries 26. Since the primary battery is not being directly used to charge the one or more capacitors 28, the primary battery need not have a high discharge rate (C rate). As a result, battery chemistries can be selected that provide an increased energy density while having a reduced discharge rate. For instance, the primary battery can have one or more cathodes that each includes a mixture of carbon monofluoride (CFx) and Silver Vanadium Oxide (SVO).

In some instances, the capacity of the primary battery 40 is the same or larger than the capacity of the secondary battery 26 in order to provide the desired charging of the secondary battery 26. In some instances, the capacity of the primary battery 40 and the secondary battery 26 is each in a range of 200 to 600 mAh.

The C rate of the secondary battery 26 can be more than 10, 50 or 100 times the C rate of the primary battery 40. The energy density of the primary battery 40 can be more than 2, 4, or 6 times the energy density of the secondary battery 26. The capacity of the primary battery 40 can be more than 1.0, 1.5, or 2.0 times the capacity of the secondary battery 26. In one example, the C rate of the secondary battery 26 is more than 10 times the C rate of the primary battery 40, the energy density of the primary battery 40 is more than 4 times the energy density of the secondary battery 26, and the capacity of the primary battery is more than 1.0 times the capacity of the secondary battery 26. In one example, the primary battery has one or more cathodes that each includes a mixture of carbon monofluoride (CFx) and Silver Vanadium Oxide (SVO) and the secondary battery is an Li-Polymer battery or a solid-state battery.

In the ICD of FIG. 1, the secondary battery 26 directly provides the electrical energy for the primary treatment charges and primary therapeutic shocks, the backup charges and backup therapeutic shocks, the maintenance charges and the maintenance shocks. Additionally, the secondary battery 26 can directly provide the electrical energy that the controller 20 uses in performing background operations such as the monitoring of the electrical characteristics of the heart, operation of the charging modules, operation of the primary charging circuit, operation of the telemetry circuit 70, operation of a storage device 21, operation of the one or more sensing circuits 22, operation of the wireless power transfer receiver 50, and detection of cardiac arrhythmias suitable for treatment by a therapeutic shock from the ICD.

In the ICD of FIG. 1, electrical energy from the primary battery 40 flows through the primary battery charging circuit 42 before flowing into the secondary battery 50 so as to charge the secondary battery 50. As a result, the primary battery 40 directly provides the electrical energy for recharging the secondary battery through the primary battery charging circuit 42. The ICD can optionally be configured such that electrical energy from the primary battery 40 flows through the primary battery charging circuit 42 can serve as at least a portion of the electrical energy in primary treatment charges, primary therapeutic shocks, backup charges, backup therapeutic shocks, maintenance charges, and maintenance shocks and/or being used to provide at least a portion of the electrical energy that powers operation of one or more background functions of the ICD. For instance, when the controller 20 concurrently operates the primary battery charging circuit 42 so as to recharge the secondary batter 26 while also performing one or more ICD functions, electrical energy from the primary battery 40 and/or the secondary battery can power the one or more ICD functions. Examples of the electrical energy powering ICD functions includes electrical energy that serve as at least a portion of the electrical energy in primary treatment charges, primary therapeutic shocks, backup charges, backup therapeutic shocks, maintenance charges, and maintenance shocks and/or being used to provide at least a portion of the electrical energy that powers operation of one or more background functions of the ICD

In the ICD of FIG. 1, the secondary battery 26 directly provides the electrical energy for a variety of ICD functions. For instance, the secondary battery 26 provides the electrical energy that becomes stored in the one or more capacitors during primary treatment charges, backup treatment charges, and maintenance charges. Additionally, the secondary battery 26 provides the electrical energy that becomes the primary therapy shocks and the backup therapy shocks. Additionally, the secondary battery 26 can provide the electrical energy that the controller 20 uses in performing background operations such as the monitoring of the electrical characteristics of the heart, operation of the charging modules, operation of the primary charging circuit, operation of the telemetry circuit 70, operation of a storage device 21, operation of the one or more sensing circuits 22, operation of the wireless power transfer receiver 50, and detection of cardiac arrhythmias suitable for treatment by a therapeutic shock from the ICD.

In the ICD of FIG. 1, electrical energy from the primary battery 40 flows through the primary battery charging circuit 42 before flowing into the secondary battery 50 so as to charge the secondary battery 50. As a result, electrical energy from the primary battery can flow through the primary battery charging circuit 42 and into the secondary battery 26 before being used in an ICD function. In some configurations of the primary battery charging circuit 42, the electrical energy from the primary battery can flow through the primary battery charging circuit 42 and be used in one or more ICD functions without flowing into or through the secondary battery. For instance, in some configurations of the primary battery charging circuit 42, electrical energy from the primary battery 40 and/or the secondary battery 26 can power one or more ICD functions when the controller 20 concurrently operates the primary battery charging circuit 42 so as to recharge the secondary batter 26 concurrently with performing the ICD function. In some configurations of the primary battery charging circuit 42, electrical energy from the primary battery 40 does not power or does not substantially the ICD functions when the controller 20 does not operate the primary battery charging circuit 42 so as to recharge the secondary battery.

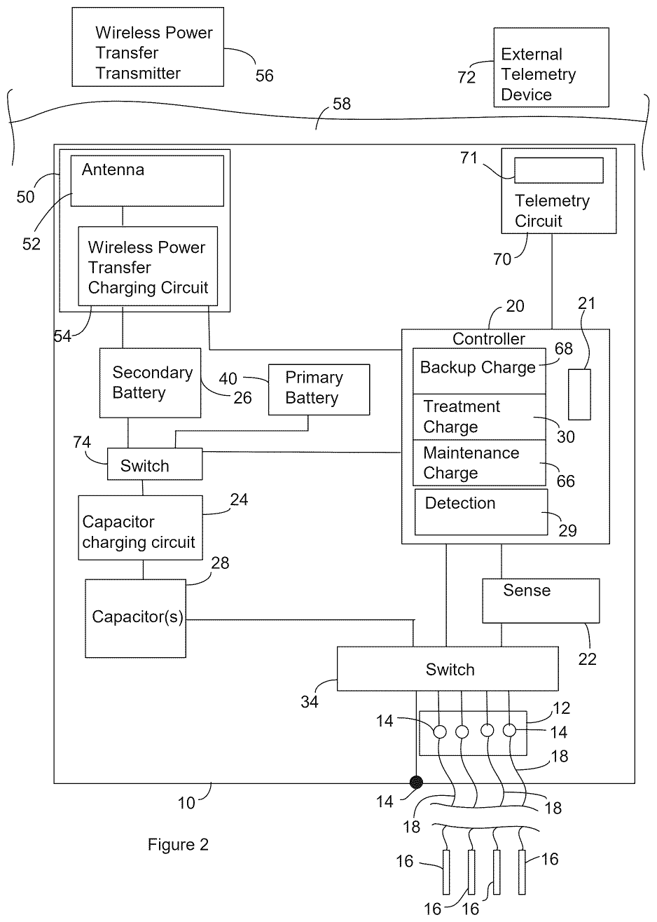

The ICD can be configured such that the power from the primary battery can be used to perform one or more ICD functions without flowing through the primary battery charging circuit 42. For instance, the ICD can be configured such that the power from the primary battery directly charges the one or more capacitors. As an example, FIG. 2 illustrates the implantable cardioverter-defibrillator (ICD) of FIG. 1 modified such that power from the primary battery can directly charge the one or more capacitors. As a result, the capacitor charging circuit 24 can charge the capacitors with electrical energy from the primary battery or from the secondary battery.

The controller 20 can select which of the multiple batteries in the ICD provides the electrical energy that charges the one or more capacitors. For instance, different portions of the multiple batteries in the ICD can be associated with different capacitor charge types. The controller 20 can perform the different types of capacitor charge such that the battery associated with the type of capacitor charge is selected as the source of the electrical energy that will charge the capacitors. As an example, a first portion of the batteries can be associated with the treatment charges and a second portion of the batteries can be associated with the maintenance charges and the backup charges. The controller 20 can perform the treatment charges such that the first portion of the batteries is selected as the source of the electrical energy that charges the capacitors for the treatment charges but the second portion of the batteries is selected as the source of the electrical energy that charges the capacitors for the backup charges and the maintenance charges.

The ICD includes a switching circuit 74 that the controller can use to select the battery that will serve as the source of electrical energy that charges the one or more capacitors. The controller 20 can operate the switching circuit 74 to select whether the primary battery 40 or the secondary battery 26 is electrically coupled with the capacitor charging circuit 24. The controller 20 can operate the capacitor charging circuit 24 such that electrical energy from the battery that is electrically coupled with the capacitor charging circuit 24 charges the one or more capacitors 28. As a result, the controller 20 can operate the switching circuit 74 in combination with the capacitor charging circuit 24 to select whether energy from the primary battery 40 or energy from the secondary battery 26 charges the one or more capacitors 28. Suitable switching circuits 74 include or consist of one or more switch components such as MOSFETs such as silicon and/or GaN MOSFETS used alone or in combination.

The controller can select the battery that charges the one or more capacitors 28 in response to the type of charging module charging the one or more capacitors 28. For instance, the controller can select the secondary battery or primary battery when the treatment module 30 charges the one or more capacitors 26 in response to the controller 20 detecting a cardiac arrhythmia that can be treated by a therapeutic shock from the ICD. Additionally, the controller can select the primary battery or the secondary battery 26 when the maintenance module 66 charges the one or more capacitors 26 in response to the controller 20 detecting satisfaction of one or more maintenance criterion. Additionally, the controller can select the primary battery or the secondary battery 26 in response to the backup module 68 charging the one or more capacitors 26 in response to the controller 20 detecting that a prior therapeutic shock has not addressed a cardiac arrhythmia.

In one example, the controller uses the primary battery to charge the one or more capacitors when the treatment module 30 charges the one or more capacitors 26 and the controller uses the secondary battery when the maintenance module 66 charges the one or more capacitors 26, and the controller uses the secondary battery when the backup module 68 charges the one or more capacitors 26.

In circumstances where the primary battery has fallen below a discharge threshold, the controller can change the battery used to charge the one or more capacitors. For instance, where the primary battery has fallen below the discharge threshold, the controller can use the secondary battery rather than the primary battery to charge the one or more capacitors when one or more of the charging modules charges the one or more capacitors 26. As a particular example, where the primary battery has fallen below the discharge threshold, the controller can use the secondary battery rather than the primary battery to charge the one or more capacitors when the treatment module 30 charges the one or more capacitors 26 in response to the controller 20 detecting a cardiac arrhythmia. Suitable discharge thresholds include, but are not limited to, state of charge thresholds. In some instances, the discharge threshold is a state of charge threshold that is less than 25%, 30% or 35%.

In embodiments of the ICD where the primary battery charges the one or more capacitors for the primary treatment charges, the secondary battery provides the electrical energy for performance of background functions such as detection of cardiac arrhythmias, for the maintenance charges, and for the backup charges. As a result, the secondary battery providing energy for the detection of cardiac arrhythmias, maintenance charges, and backup charges can be recharged using the wireless power transfer transmitter 56 and the wireless power transfer receiver 50. Since the energy that provides these functions can be replaced over time, the frequency of primary treatment charges can determine the life of the ICD before surgical intervention is needed for maintenance of the ICD. The primary battery can be selected to have a capacity that provides a particular number of defibrillation shocks over the life of the primary battery. The number of defibrillation shocks that can be provided by the primary battery can determine the amount of time that can pass before surgery for maintenance of the ICD is required In one example, the primary battery has a capacity suitable for providing 20 defibrillation shocks that are each at 870V and 48J. An example of a suitable primary battery is a battery that includes or consists of silver vanadium oxide (SVO) as an active material in one or more cathodes.

In the ICD embodiment of FIG. 2, a suitable voltage for the primary battery 40 includes, but is not limited to, a beginning of service (BOS) voltage greater than 1.5V, 2.2V, or 2.5V and less than 4.35V, 4.25V or 3.25V and a suitable voltage for the secondary battery includes, but is not limited to, a fully charge voltage at beginning of service that is greater than 3.2V, 3.5V, or 3.7V and less than 4.35V, 4.25V, or 4.0V. The C rate of the secondary battery 26 can be more than 10, 50, or 100 times the C rate of the primary battery 40. The energy density of the primary battery 40 can be more than 2, 4, or 6 times the energy density of the secondary battery 26. The capacity of the primary battery 40 can be more than 1.0, 1.5, or 2.0 times the capacity of the secondary battery 26. In one example, the C rate of the secondary battery 26 is more than 10 times the C rate of the primary battery 40, the energy density of the primary battery 40 is more than 4 times the energy density of the secondary battery 26, and the capacity of the primary battery is more than 1.1 times the capacity of the secondary battery 26. In one example, the primary battery has a cathode that includes a mixture of carbon monofluoride (CFx) and Silver Vanadium Oxide (SVO) and the secondary battery is an Li-Polymer battery or a solid-state battery.

In some instances, a patient having an ICD constructed according to FIG. 1 or FIG. 2 can be scheduled for one or more visits to a clinic for maintenance of the ICD. A clinic can be characterized by the presence of one or more medical personnel, healthcare personnel, or technicians that are familiar with the operation, maintenance, and/or monitoring of the ICD and who are at least occasionally present at the clinic at least partially for the purpose of operating, maintaining, and/or monitoring of ICDs. The scheduled visits can be periodic or substantially periodic. For instance, a period of time between scheduled visits can be less than or equal to the time threshold disclosed in the context of the one or more maintenance criteria. A maintenance charge can be performed during the visit to the clinic. For instance, a transmitter external to the ICD and the patient can transmit to the ICD a request for the ICD to perform a maintenance charge. As an example, during a visit to the clinic, an external telemetry device 72 in wireless communication with the telemetry circuit 70 can transmit to the telemetry circuit 70 a signal that the controller 20 and/or the maintenance module 66 recognizes as a request for performance of a maintenance charge. In response, the maintenance module 66 can perform the requested maintenance charge. As a result, receipt of the signal recognized as a request for performance of a maintenance charge can serve as the satisfaction of the one or more maintenance criteria. Accordingly, the maintenance charge(s) performed at the clinic can be in addition to the maintenance charge(s) performed in response to the satisfaction of the one or more maintenance criteria described above or an alternative to the one or more maintenance criteria described above.

When the ICD is constructed according to FIG. 1, the secondary battery 26 directly provides the electrical energy for the maintenance charge. When the ICD is constructed according to FIG. 2, the controller selects the secondary battery to directly provides the electrical energy for the maintenance charge. During and/or after a maintenance charge performed as part of a visit to the clinic, the controller 20 can operate the wireless power transfer charging circuit 54 so as to charge the secondary battery 26 from an external power source such as the wireless power transfer transmitter 56. For instance, the controller 20 can operate the wireless power transfer charging circuit 54 such that the secondary battery 26 is charged using power received from the wireless power transfer transmitter 56. In some instances, the secondary battery is recharged such that the secondary battery stays within the characteristic window such as the state of charge window. For instance, the controller 20 can stop charging the secondary battery 26 in response to the state of charge increasing above the upper threshold of the characteristic window. The recharge of the secondary battery can be started and/or completed before the patient leaves the clinic. For instance, the recharge of the secondary battery can be started and/or completed within 2 or 6 hours of the completion of the maintenance charge. As a result, maintenance charges can be performed on a schedule and at least the amount of energy removed from the secondary battery can be replaced.

The performance of scheduled maintenance charges at a clinic does not significantly deplete the energy available from the primary battery and the secondary battery because the energy used to perform the maintenance charge is replaced. In prior ICD, 20 to 50% of the battery capacity is consumed performing maintenance charges. Due to the high energy requirements associated with maintenance charges, the capacitors in prior ICDs are often charged to the treatment charge voltage only once or were charged to less than the treatment charge voltage during maintenance charges. Since the energy used in the maintenance charges can now be replaced, the energy demands of the maintenance charges allow the capacitors in prior ICDs to be charged to at least the treatment charge voltage multiple times and/or to be held at the treatment charge voltage for the charge hold time. Additionally, the size of the capacitors can be reduced because of reduced levels of deformation that occur as a result of the scheduled maintenance charges.

In the above description of the ICD operation during a visit to the clinic, the ICD has access to the external telemetry device 72 and to the wireless power transfer transmitter 56. As a result, the wireless power transfer transmitter 56 and/or the external telemetry device 72 can be a stand-alone component or can be included in or on an article of clothing or equipment worn by the patient. For instance, the wireless power transfer transmitter 56 and the external telemetry device 72 can be included in or on a vest worn by the patient. When the wireless power transfer transmitter 56 and the external telemetry device 72 are included in or on an article of clothing or equipment worn by the patient, the article of clothing or equipment can hold the wireless power transfer transmitter 56 in an appropriate location relative to the wireless power transfer receiver 50 to achieve wireless power transfer and can hold the external telemetry device 72 in an appropriate location relative to the telemetry circuit 70 in order to permit the exchange of signals between the external telemetry device 72 and the telemetry circuit 70.

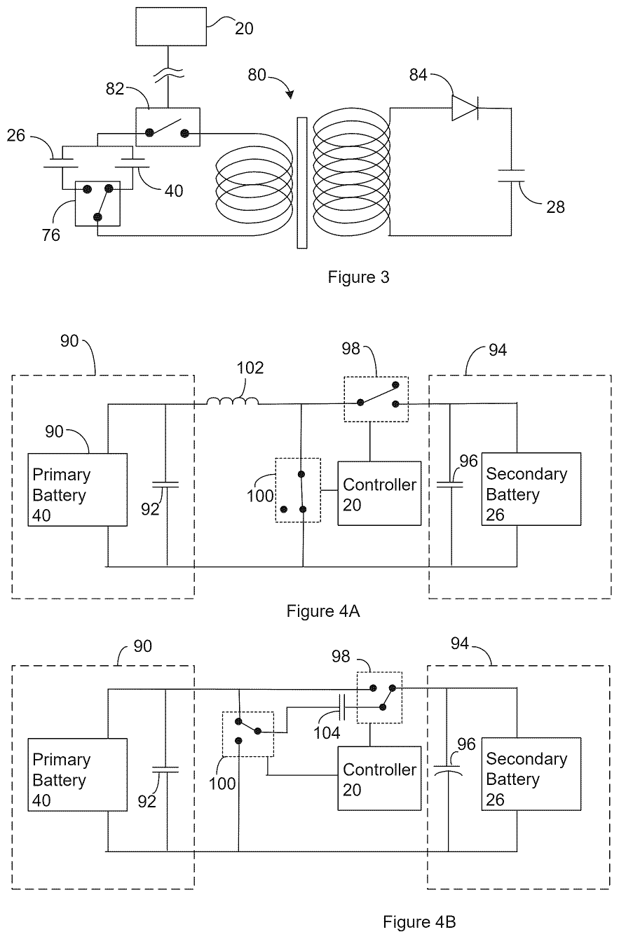

Suitable capacitor charging circuits 24 include, but are not limited to, electric power converters such as DC-to-DC converters and switched DC-to-DC converters. FIG. 3 is a schematic of an example of a suitable capacitor charging circuit 24. For the purpose of illustration, the capacitor charging circuit 24 of FIG. 3 is shown with the primary battery 42 and secondary battery 26 arranged as shown in FIG. 2. The capacitor charging circuit 24 illustrated in FIG. 3 can be used with the secondary battery 26 arrangement shown in FIG. 1. For instance, the primary battery 42, secondary battery 26, and switching circuit 74 shown in FIG. 3 can be replaced with a secondary battery 26 to provide the arrangement of FIG. 1.

The capacitor charging circuit 24 includes a transformer 80. Suitable transformers 80 include, but are not limited to, a DC-to-DC transformer or fly back transformer. The capacitor charging circuit 24 includes a switch 82 that is electrically connected between the transformer 80 and the primary battery 42 or secondary battery 26. Additionally, the capacitor charging circuit 24 includes a rectifying diode 84 electrically connected between the transformer 80 and the one or more capacitors 28. The charging modules can charge the one or more capacitors 28 by rapidly opening and closing the switch 82 so as to generate an AC current that is input to the transformer 80. The rectifying diode 84 receives the AC current from the transformer 80 and outputs a DC current that charges the one or more capacitors 28. Suitable switches 82 include, but are not limited to, insulated gate bipolar transistors (IGBTs) and MOSFETs such as silicon and/or GaAs MOSFETS used alone or in combination.

FIG. 4A is a schematic of an example of a boost converter suitable for use as the primary battery charging circuit 42. The illustrated boost converter includes a primary battery branch 90 where the primary battery 42 is connected in parallel with a first capacitor 92. The illustrated boost converter includes a secondary battery branch 94 where the secondary battery 26 is connected in parallel with a second capacitor 96. A first switch 98 is connected in series with the secondary battery branch 94. A second switch 100 is connected in parallel with the combination of the serially connected first switch 98 and the secondary battery branch 94. A node Suitable switches for use as the first switch 98 and/or the second switch 100 include, but are not limited to, transistors. An inductor 102 is positioned along an electrical pathway between the primary battery branch 90 and the first switch 98. The controller 20 can operate the first switch 98 and the second switch 100. In order to charge the secondary battery 26, the controller alternates first cycles where the first switch 98 is off and the second switch 100 is on with second cycles where the first switch 98 is on and the second switch 100 is off. During the first cycles, the inductor 102 generates an electrical field and stores energy. During the second cycles, the magnetic field collapses and creates a voltage across the secondary battery 26.

FIG. 4B is a schematic of an example of a capacitive charge pump suitable for use as the primary battery charging circuit 42. The illustrated capacitive charge pump includes the primary battery branch 90 and the secondary battery branch 94 disclosed in the context of FIG. 4A. A first switch 98 is positioned along an electrical pathway between the primary battery branch 90 and the secondary battery branch 94. The controller 20 can operate the first switch 98 in a first mode where first switch 98 creates an electrical pathway between the secondary battery branch 94 and a capacitor 104 or in a second mode where the first switch 98 creates an electrical pathway between the secondary battery branch 94 and the primary battery branch 90. The primary battery branch 90 is electrically coupled with a second switch 100. When the controller 20 creates the electrical pathway between the secondary battery branch 94 and the primary battery branch 90, an electrical pathway between the secondary battery branch 94 and the second switch 100 is also created The capacitor 104 is also electrically coupled with the second switch 100. The controller can operate the second switch 100 in a first mode where the second switch 100 creates an electrical pathway between the capacitor 104 and the primary battery branch 90 or a second mode where the second switch 100 creates an electrical pathway between the capacitor 104 and the ground side of both the primary battery branch 90 and the secondary battery branch 94.

In order to charge the secondary battery 26, the controller alternates first cycles where the second switch 100 is operated in the second mode and the first switch 98 is operated in the first mode with second cycles where the second switch 100 is operated in the first mode and the first switch 98 is operated in the second mode. During the first cycles, the primary battery charges the capacitor 104. During the second cycles, the capacitor 104 is connected in series with the primary battery branch 90 and the combination of the capacitor 104 and primary battery branch 90 are connected in parallel with the secondary battery branch 94. As a result, the serially connected capacitor 104 and primary battery branch 90 charge the secondary battery 26.

FIG. 5 is a schematic of an example of a basic wireless power transfer system that includes a wireless power transfer receiver and a wireless power transfer transmitter suitable for use as the wireless power transfer receiver 50 and the wireless power transfer transmitter 56. The wireless power transfer transmitter 56 includes a coil driver 106 electrically coupled with an oscillator 108 and a capacitively loaded transmitter coil 110. The coil driver 108 is configured to place oscillating energy from the oscillator 106 onto the transmitter coil 110 such that the transmitter coil 110 generates an oscillating magnetic field.

The wireless power transfer receiver 50 includes a capacitively loaded receiver coil 112 that can be magnetically coupled with the transmitter coil 110 so as to provide near field wireless transmission of electrical energy between magnetically coupled coils. The wireless power transfer receiver 50 can include a rectifier 114 to generate a DC voltage from the receiver coil 112. The resulting DC voltage can be used to charge the secondary battery 26. In addition to the rectifier 114 or as an alternative to the rectifier 114, a regulator circuit (not shown) can be positioned on the electrical pathway between the receiver coil 112 and the secondary battery 26 to regulate the movement of the electrical energy from the receiver coil 112 to the secondary battery 26. The controller 20 can operate one or more optional switches 116 so as to connect and disconnect the secondary battery 26 from charging by the wireless power transfer receiver 50 as needed. In the wireless power transfer system, the receiver coil 112 can serve as the antenna 52 and the rectifier can serve as a component in the wireless power transfer charging circuit 54.

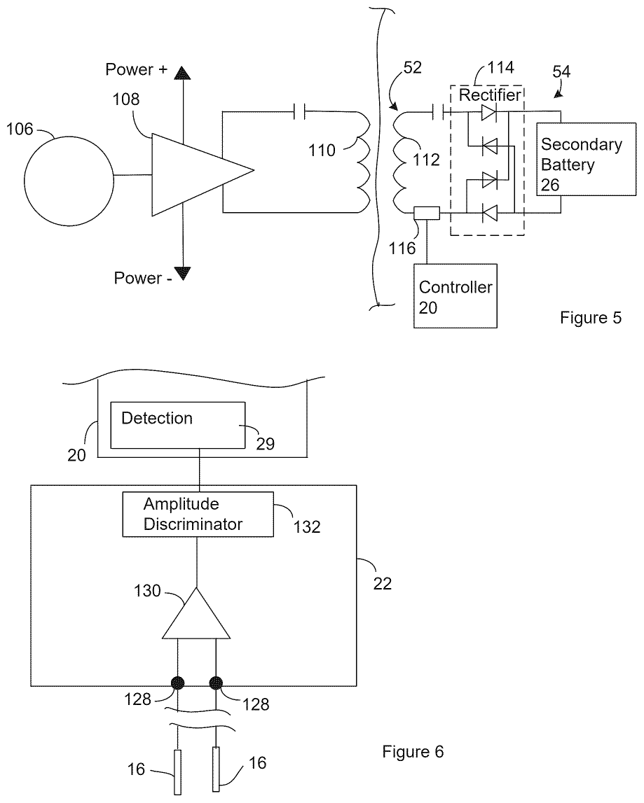

FIG. 6 is a diagram that includes a schematic of an example of a sensing circuit 22. The sensing circuit 22 includes inputs 128. The switch 34 shown in FIG. 1 and FIG. 2 can be operated such that each of the inputs 128 is electrically coupled with an electrode 16 that is configured to output electrical signals that indicate the presence of a cardiac arrhythmia. For instance, the electrical signals output from the electrodes 16 are electrocardiogram (ECG) signals. The sensing circuit 22 includes an amplifier 130 that receives the electrical signals from the electrodes 16 and outputs amplified electrical signals. Suitable amplifiers include, but are not limited to, operational amplifiers (op amps). The sensing circuit 22 includes amplitude discriminator 132 that receives the amplified electrical signals and outputs a data signal that is received by the detection module 29.

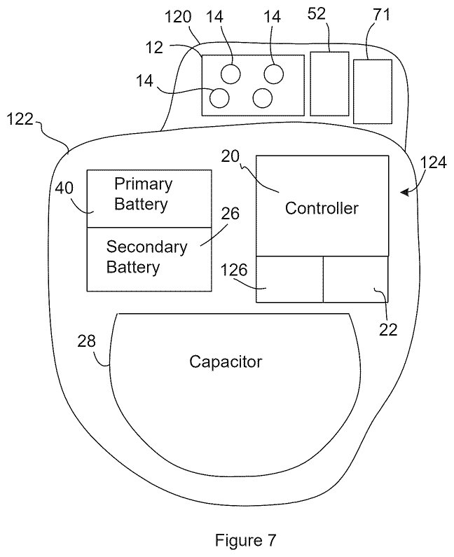

FIG. 7 is a diagram of an example of a pulse generator for use in an ICD. The container 10 includes a header 120 mounted on a housing 122 that is often referred to as the “canister,” “can,” “case,” or “case electrode.” The header 120 includes the connector block 12 with the one or more terminals 14. The header 120 also includes the antenna 52 for the wireless power transfer receiver 50 and the antenna 71 for the telemetry circuit 70. The antenna 52 for the wireless power transfer receiver 50 and the antenna 71 for the telemetry circuit 70 can be different. In some instances, the same antenna can be used as the antenna 52 for the wireless power transfer receiver 50 and as the antenna 71 for the telemetry circuit 70.

The primary battery 40, the secondary battery 26, and the one or more capacitors 28 are positioned in the housing 122. Electronics 124 that operate the ICD are also positioned in housing. The electronics include the controller 20, one or more sensing circuits 22, and the charging circuits 126. The charging circuits 126 can include the capacitor charging circuit 24, the wireless power transfer charging circuit 54, and the primary battery charging circuit 42.

The time for replacement of the primary battery in the disclosed ICDs can be a pre-determined time period or can be based on data that the ICD transits from the telemetry circuit 70 to the external telemetry device 72.

The controller 20 can include additional components such as logic and timing circuitry, state machine circuitry, and I/O circuitry. Additionally, the ICD can optionally perform additional functions and operations. For instance, the controller 20 can optionally provide pacing functionality using the disclosed electrodes and/or additional electrodes electrically coupled with the controller. As an example, the controller 20 can provide bradycardia prevention, tachycardia, atrial pacing, rate-responsive pacing and atrial anti-tachycardia pacing and defibrillation.

Although the ICD is disclosed as having one primary battery and one secondary battery, the ICD can include multiple primary batteries in place of the primary battery and/or multiple secondary batteries in place of the secondary battery.

In some instances, the secondary batteries in the above ICDs can be distinguished from the primary batteries because at least one time after the secondary battery is discharged to below 50% of the battery capacity, the secondary battery can be recharged to more than 75% of the capacity.

Other embodiments, combinations and modifications of this invention will occur readily to those of ordinary skill in the art in view of these teachings. Therefore, this invention is to be limited only by the following claims, which include all such embodiments and modifications when viewed in conjunction with the above specification and accompanying drawings.

1. An implantable cardioverter-defibrillator (ICD), comprising:
one or more capacitors;
a secondary battery and a primary battery;
a charging circuit electrically coupled to the primary battery and the secondary battery;
a capacitor charging circuit electrically coupled to the one or more capacitors and the secondary battery; and
a controller configured to operate the capacitor charging circuit such that the one or more capacitors are charged with electrical energy from the secondary battery, and
the controller being configured to operate the charging circuit such that electrical energy from the primary battery recharges the secondary battery.

2. The implantable cardioverter-defibrillator (ICD) of claim 1, wherein a beginning of service voltage of the primary battery is below a voltage of the secondary battery at a 100% state of charge.

3. The implantable cardioverter-defibrillator (ICD) of claim 1, further comprising:
a wireless power transfer receiver configured to receive electrical energy from a wireless power transfer transmitter located outside of a patient while the ICD is located inside of the patient;
the wireless power transfer receiver including a wireless power transfer charging circuit; and
the controller being configured to operate the wireless power transfer charging circuit such that the secondary battery is recharged with the electrical energy that the wireless power transfer receiver receives from the wireless power transfer transmitter.

4. An implantable cardioverter-defibrillator (ICD), comprising:
one or more capacitors;
multiple batteries electrically coupled with the one or more capacitors;
a capacitor charging circuit electrically coupled to the one or more capacitors and the multiple batteries; and
a controller being configured to switchably select a battery as a source for electrical energy that charges the one or more capacitors, the selected battery being selected from among the multiple batteries.

5. The implantable cardioverter-defibrillator (ICD) of claim 4, wherein the multiple batteries include at least one primary battery and at least one secondary battery.

6. The implantable cardioverter-defibrillator (ICD) of claim 4, wherein the controller is configured to perform capacitor charges where the controller operates the capacitor charging circuit such that the one or more capacitors are charged with electrical energy from the selected battery and the one or more capacitors are not charged with electrical energy from a portion of the multiple batteries that are not the selected battery.

7. The implantable cardioverter-defibrillator (ICD) of claim 4, wherein the controller is configured to perform capacitor charges where the controller operates the capacitor charging circuit such that the one or more capacitors are charged with electrical energy from the selected battery,
the capacitor charges include multiple different types of capacitor charge, and
the controller configured to select a different one of the multiple batteries for different types of capacitor charge.

8. The implantable cardioverter-defibrillator (ICD) of claim 7, wherein the different types of capacitor charge include maintenance charges and treatment charges,
the controller being configured to perform the maintenance charges response to a time since the one or more capacitors were last charged exceeding a time threshold,
the controller being configured to perform the maintenance charges in response to the controller detecting occurrence of a cardiac arrhythmia suitable for treatment by the ICD, and
the controller being configured to select a different one of the multiple batteries for the maintenance charges than is selected for the treatment charges.

9. The implantable cardioverter-defibrillator (ICD) of claim 8, wherein the multiple batteries include a primary battery and a secondary battery and the primary battery is the selected battery during the treatment charges and the secondary battery is the selected battery during the maintenance charges.

10. The implantable cardioverter-defibrillator (ICD) of claim 4, further comprising:
a wireless power transfer receiver configured to receive electrical energy from a wireless power transfer transmitter located outside of a patient while the ICD is located inside of the patient;
the wireless power transfer receiver including a wireless power transfer charging circuit; and
the controller being configured to operate the wireless power transfer charging circuit such that the secondary battery is recharged with the electrical energy that the wireless power transfer receiver receives from the wireless power transfer transmitter.

11. A method of operating an implantable cardioverter-defibrillator (ICD), comprising:
selecting from among multiple batteries a source battery; and
charging one or more capacitors with electrical energy from the source battery.

12. The method of claim 11, wherein electrical energy from the portion of the multiple batteries that are not selected as the source battery does not charge the one or more capacitors while charging one or more capacitors with the electrical energy from the source battery.

13. An implantable cardioverter-defibrillator (ICD), comprising:
one or more capacitors;
a telemetry receiver configured to be in wireless communication with a telemetry transmitter located outside of a patient while the ICD is located inside of the patient; and
a controller configured to perform a maintenance charge of the one or more capacitors in response to the telemetry receiver receiving from the telemetry transmitter a request that the controller perform the maintenance charge,
the maintenance charge being such that electrical energy charges the one or more capacitors during the maintenance charge.

14. The ICD of claim 13, wherein the ICD is configured to be electrically coupled with one or more electrodes implanted within the patient and to provide electrical energy stored in the one or more capacitors during a treatment charge of the one or more capacitors to the one or more electrodes so as to provide a therapeutic shock to the patient but the electrical energy that charges the one or more capacitors during the maintenance charge is not provide from the one or more capacitors to the one or more electrodes.

15. The ICD of claim 13, further comprising: a secondary battery and a primary battery; and
the maintenance charge being performed such that electrical energy from the secondary battery directly charges the one or more capacitors during the maintenance charge.

16. The ICD of claim 13, wherein the controller is configured to operate a switch circuit so as to select the primary battery or the secondary battery as a source for the electrical energy that charges the one or more capacitors.

17. The ICD of claim 16, wherein the controller is configured to select the secondary battery as the source for the electrical energy that charges the one or more capacitors during the maintenance charge.

18. The ICD of claim 13, further comprising:
a wireless power transfer receiver configured to receive electrical energy from a wireless power transfer transmitter located outside of a patient while the ICD is located inside of the patient;
the wireless power transfer receiver including a wireless power transfer charging circuit; and
the controller being configured to operate the wireless power transfer charging circuit such that the secondary battery is recharged with the electrical energy that the wireless power transfer receiver receives from the wireless power transfer transmitter.

19. A method of operating an implantable cardioverter-defibrillator (ICD), comprising:
transmitting a signal from a telemetry transmitter located outside of a patient to the ICD while the ICD is located inside of the patient, the signal causing the ICD to perform a maintenance charge of one or more capacitors included in the ICD;
transmitting electrical energy from a wireless power transfer transmitter to a wireless power transfer receiver included in the ICD while the ICD is located inside of the patient, the ICD employing the electrical energy received from the wireless power transfer transmitter to recharge a secondary battery included in the ICD; and
the signal and the electrical energy being transmitted during the same visit of the patient to a treatment clinic.

20. The method of claim 19, wherein the electrical energy is transmitted within 6 hours of the signal being transmitted.
